SEQUENCE LISTING (10 sequences)

SEQ ID NO: 1 - DNA, 57 bp, Artificial Sequence
  source: /note=“Description of Artificial Sequence Synthetic oligonucleotide”
atgggctggtcctgcatcatcctgttcctggtggcaaccgcaacaggagtgcacagc

SEQ ID NO: 2 - DNA, 1365 bp, Artificial Sequence
  source: /note=“Description of Artificial Sequence Synthetic polynucleotide”
caggtgcagctgcagcagtggggagcaggactgctgaagccttctgagaccctgagcctgacatgtgccgtgtatggcggcagcttttccggctactattggtcctggatcaggcagccacctggcaagggactggagtggatcggcgagatcaaccactctggcagcaccaactacaatccctctctgcggagcagagtgaccatctccgtggacacatctaagaatcagttctctctgaagctgcgcagcgtgaccgcagcagatacagccgtgtactattgcgccaggggctttatggtgcgcggcatcatgtggaactactattacatggacgtgtggggcaagggcaccacagtgaccgtgtccccatctgccagcacaaagggaccaagcgtgttccctctggcaccaagctccaagtccacctctggaggaacagccgccctgggctgtctggtgaaggattatttccctgagccagtgaccgtgtcctggaactctggcgccctgacctccggagtgcacacatttccagccgtgctgcagtctagcggcctgtatagcctgtcctctgtggtgaccgtgcccagctcctctctgggcacccagacatacatctgcaacgtgaatcacaagccaagcaatacaaaggtggacaagcgggtggagcccaagtcctgtgataagacccacacatgcccaccatgtccagcacctgagctgctgggaggaccaagcgtgttcctgtttcctccaaagcctaaggacaccctgatgatctctagaacccccgaggtgacatgcgtggtggtggacgtgagccacgaggatcctgaggtgaagttcaactggtacgtggatggcgtggaggtgcacaatgccaagaccaagccccgggaggagcagtataactccacctacagagtggtgtctgtgctgacagtgctgcaccaggactggctgaacggcaaggagtacaagtgcaaggtgtccaataaggccctgccagcccccatcgagaagaccatctctaaggcaaagggacagccaagggagcctcaggtgtatacactgcccccttcccgcgacgagctgaccaagaaccaggtgtctctgacatgtctggtgaagggcttttacccttctgatatcgccgtggagtgggagagcaatggccagccagagaacaattataagaccacaccacccgtgctggacagcgatggctccttctttctgtacagcaagctgaccgtggataagtcccggtggcagcagggcaacgtgttcagctgctccgtgatgcacgaggccctgcacaatcactacacccagaagtctctgagcctgtcccctggcaag

SEQ ID NO: 3 - DNA, 123 bp, Artificial Sequence
  source: /note=“Description of Artificial Sequence Synthetic polynucleotide”
cacgagaccacaccaaataagggcagcggcaccacatccggcaccacaagactgctgagcggccacacctgttttaccctgacaggcctgctgggcaccctggtgacaatgggcctgctgaca

SEQ ID NO: 4 - DNA, 57 bp, Artificial Sequence
  source: /note=“Description of Artificial Sequence Synthetic oligonucleotide”
atggcctggacccctctgtggctgacactgtttaccctgtgcatcggctctgtggtg

SEQ ID NO: 5 - DNA, 642 bp, Artificial Sequence
  source: /note=“Description of Artificial Sequence Synthetic polynucleotide”
agctccgagctgacacaggacccagtggtgagcgtggccctgggacagacagtgcggatcacctgtcagggcgattctctgagaacctaccacgccagctggtatcagcagaagccaaggcaggcccccgtgctggtcatctacgacgagaacaataggccttccggcatcccagatcgcttctccggctctacaagcggcaacaccgcctctctgacaatcaccggagcacaggcagaggacgaggcagattactattgcaactcccgggactctagcggcaatagactggtgttcggaggaggaacaaagctgaccgtgctgggacagccaaaggcagcaccttccgtgaccctgtttccaccttcctctgaggagctgcaggccaataaggccaccctggtgtgcctgatcagcgacttctacccaggagcagtgacagtggcatggaaggccgatagctccccagtgaaggccggcgtggagaccacaacccccagcaagcagtccaacaataagtacgccgcctctagctatctgtccctgacccccgagcagtggaagtctcacagatcctattcttgccaggtgacacacgagggcagcacagtggagaagaccgtggcccctacagagtgttcc

SEQ ID NO: 6 - protein, 19 aa, Artificial Sequence
  source: /note=“Description of Artificial Sequence Synthetic peptide”
MGWSCIILFLVATATGVHS

SEQ ID NO: 7 - protein, 455 aa, Artificial Sequence
  source: /note=“Description of Artificial Sequence Synthetic polypeptide”
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 41 aa, Artificial Sequence
  source: /note=“Description of Artificial Sequence Synthetic polypeptide”
HETTPNKGSGTTSGTTRLLSGHTCFTLTGLLGTLVTMGLLT

SEQ ID NO: 9 - protein, 19 aa, Artificial Sequence
  source: /note=“Description of Artificial Sequence Synthetic peptide”
MAWTPLWLTLFTLCIGSVV

SEQ ID NO: 10 - protein, 214 aa, Artificial Sequence
  source: /note=“Description of Artificial Sequence Synthetic polypeptide”
SSELTQDPVVSVALGQTVRITCQGDSLRTYHASWYQQKPRQAPVLVIYDENNRPSGIPDRFSGSTSGNTASLTITGAQAEDEADYYCNSRDSSGNRLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2023/0002785 A1
(43) Pub. Date: Jan. 5, 2023

(54) MRNA-ENCODED ANTIBODIES FOR CONTRACEPTION

(71) Applicants: GEORGIA TECH RESEARCH CORPORATION, Atlanta, GA (US); ZABBIO, INC., San Diego, CA (US)

(72) Inventors: Philip J. SANTAGELO, Smyrna, GA (US); Daryll VANOVER, Duluth, GA (US); Kevin J. WHALEY, San Diego, CA (US)

(73) Assignees: GEORGIA TECH RESEARCH CORPORATION, Atlanta, GA (US); ZABBIO, INC., San Diego, CA (US)

(21) Appl. No.: 17/772,932

(22) PCT Filed: Oct. 28, 2020
(86) PCT No.: PCT/US2020/057713

§ 371 Date: Apr. 28, 2022

Related U.S. Application Data
(60) Provisional application No. 62/926,771, filed on Oct. 28, 2019.

(51) Int. Cl. C12N 15/86; C07K 16/28; A61K 35/54; A61K 9/00; A61P 15/18; A61K 9/12

(52) U.S. Cl. CPC C12N 15/86; C07K 16/2893; A61K 35/54; A61K 9/0034; A61P 15/18; A61K 9/12; C07K 2319/035; A61K 2039/505

(57) ABSTRACT

Non-hormonal contraception compositions and methods for contraception are provided. One embodiment provides an antibody or an antigen binding fragment thereof that specifically binds to one or more sperm antigens and inhibits the ability of anti-body-bound sperm to fertilize an egg. Typically, the antibody is a monoclonal antibody, for example a human or humanized monoclonal antibody. In one embodiment, the antibody or antigen binding fragment thereof specifically binds to CD52g expressed on vertebrate, for example human, sperm cells and inhibits, blocks, or reduces the ability of the antibody-bound sperm to fertilize an egg. In one embodiment the antibody contains a membrane anchor. The membrane anchor can contain transmembrane domains, glycosylphosphatidylinositol anchors, or myristoylation motifs.

### CROSS-REFERENCE TO RELATED APPLICATIONS

This application claims benefit of and priority to U.S. Provisional Patent Application No. 62/926,771 filed on Oct. 28, 2019, and is incorporated by reference in its entirety.

### STATEMENT REGARDING FEDERALLY SPONSORED RESEARCH

This invention was made with government support under R61HD099745 awarded by the National Institutes of Health. The government has certain rights in the invention.

### TECHNICAL FIELD OF THE INVENTION

Aspects of the invention are directed to compositions and methods for contraception.

### BACKGROUND OF THE INVENTION

In the US, 45% of pregnancies were unintended in 2011. The vast majority of unintended pregnancy occurred when contraception was used inconsistently or not at all. Currently 72% of women who practice contraception use hormonal methods, but there is frequent dissatisfaction with these methods, due to quality of life and safety concerns; a recent high profile study (March, L S, et al., N Engl J Med., 7; 377(23):2228-39 (2017)), brought the risk of breast cancer back to the discussion.

Currently, contraception is achieved by either physical blockage of the fallopian tube (through intrauterine devices) or hormonal therapy (such as Depo-Provera®, Loestrin®, or Ortho Evra®). However, both of these contraception strategies have drawbacks. IUDs can cause severe pain and result in infections and other complications. Hormones can have unwanted side effects such as weight gain, increased formation of blood clots, headaches, nausea, etc.

Reversible immunocontraception offers a non-hormonal solution, where antibodies are introduced into the female reproductive tract (FRT) and inhibit sperm function. This approach, though, has a number of challenges including: identification of a specific and effective monoclonal antibody (Ab) against a human sperm antigen, and a safe and reliable method for introduction of Abs that is temporally and spatially controllable.

Therefore, there is a clear need for new approaches to non-hormonal female contraceptives that are easy to use, woman-applied, and have a controllable duration of action.

### SUMMARY OF THE INVENTION

Non-hormonal contraception compositions and methods for contraception are provided.

One embodiment provides an antibody or an antigen binding fragment thereof that specifically binds to one or more sperm antigens and inhibits the ability of antibody-bound sperm to fertilize an egg. Typically, the antibody is a monoclonal antibody, for example a human or humanized monoclonal antibody. In one embodiment, the antibody or antigen binding fragment thereof specifically binds to CD52g expressed on vertebrate, for example human, sperm cells and inhibits, blocks, or reduces the ability of the antibody-bound sperm to fertilize an egg. In one embodiment the antibody contains a membrane anchor. The membrane anchor can contain transmembrane domains, glycosylphosphatidylinositol anchors, or myristoylation motifs.

Another embodiment provides a recombinant genetic construct. The construct encodes an antibody or antigen binding fragment thereof that specifically binds to a sperm antigen and a membrane anchor. The genetic construct can be configured to be delivered and expressed in an animal subject, for example a human. In one embodiment the genetic construct is an RNA construct including but not limited to a mRNA construct.

Another embodiment provides a therapeutic mRNA that expresses an antibody or antigen binding fragment there that specifically binds to sperm and inhibits antibody-bound sperm for fertilizing an egg. In some embodiments, the antibody is an immunoglobulin G, immunoglobulin M, immunoglobulin A, immunoglobulin D, or immunoglobulin E. In one embodiment the antibody specifically binds to CD52g expressed on sperm cells. In some embodiments the antibody contains a membrane anchor. The membrane anchor can contain transmembrane domains, glycosylphosphatidylinositol anchors, or myristoylation motifs.

Another embodiment provides a pharmaceutical composition containing a nucleic acid construct encoding an antibody or antigen binding fragment thereof that specifically binds to a sperm antigen and a membrane anchor. In one embodiment the nucleic construct is an mRNA construct, for example an mRNA construct. In one embodiment the sperm antigen is CD25g. In some embodiments, the pharmaceutical composition contains an excipient. In some embodiments, the excipient is water. The membrane anchor can contain transmembrane domains, glycosylphosphatidylinositol anchors, or myristoylation motifs. In one embodiment, pharmaceutical contains anti-CD52g antibodies.

One embodiment provides a method for providing contraception to a female subject in need thereof including the steps of administering to the subject's female reproductive tract a nucleic acid construct encoding an antibody or an antigen binding fragment thereof and a membrane anchor in an amount effective to provide contraception. In one embodiment, the nucleic acid construct is a mRNA construct. In some embodiments the construct is delivered as an aerosol. In other embodiments, the construct is delivered using nanoparticles, for example lipid nanoparticles containing polyethylenimine (PEI) or modified PEI. In some embodiments the construct can be delivered using poly-beta-amino-esters nano-vehicles (PBAEs), and modified PBAEs.

Another embodiment provides a method for providing contraception to a female subject in need thereof by transfecting FRT epithelial cells with a nucleic acid construct encoding an antibody or an antigen-binding fragment thereof that specifically binds to a sperm antigen and also encodes a membrane anchor in an amount effective to provide contraception.

One embodiment provides a kit containing a nucleic acid construct encoding an antibody or an antigen-binding fragment thereof that specifically binds to a sperm antigen and also encodes a membrane anchor, and a delivery device. In some embodiments the delivery device is and atomizer or a dual-chamber syringe containing lyophilized mRNA and water (allowing for cold-chain independence), and an atomizer suitable for self-insertion into the FRT.

In one embodiment, the complete HCA Heavy Chain mRNA contains a signal sequence, heavy chain sequence, and, if included, membrane anchor sequence.

One embodiment provides a vector having a nucleic acid encoding a signal sequence having 85%, 90%, 95%, 99%, or 100% sequence identity to SEQ ID NO:1.

One embodiment provides an antibody or antigen binding fragment thereof having a heavy chain encoded by a nucleic acid sequence having 85%, 90%, 95%, 99%, or 100% sequence identity SEQ ID NO:2.

One embodiment provides an antibody or an antigen binding fragment thereof containing a GPI membrane anchor encoded by a nucleic acid sequence having 85%, 90%, 95%, 99%, or 100% sequence identity to SEQ ID NO:3.

In one embodiment the complete HCA Light Chain mRNA contains a signal sequence and a light chain sequence.

One embodiment provides a vector containing a nucleic acid encoding a signal sequence encoded by a nucleic acid having 85%, 90%, 95%, 99%, or 100% sequence identity to the following sequence SEQ ID NO:4.

One embodiment provides an antibody or an antigen binding fragment thereof having a light chain encoded by a sequence having 85%, 90%, 95%, 99%, or 100% sequence identity to SEQ ID NO:5.

One embodiment provides an antibody having a heavy chain encoded by a nucleic acid sequence having 85%, 90%, 95%, 99%, or 100% sequence identity SEQ ID NO:2, a GPI membrane anchor encoded by a nucleic acid sequence having 85%, 90%, 95%, 99%, or 100% sequence identity to SEQ ID NO:3, and a light chain encoded by a sequence having 85%, 90%, 95%, 99%, or 100% sequence identity to SEQ ID NO:5.

Another embodiment provides a recombinant genetic construct or vector. The construct encodes an antibody or antigen binding fragment thereof that specifically binds to a sperm antigen and a membrane anchor. The genetic construct can be configured to be delivered and expressed in an animal subject, for example a human. In one embodiment, the recombinant genetic vector includes a nucleic acid encoding a heavy chain encoded by a nucleic acid having 85%, 90%, 95%, 99%, or 100% sequence identity to SEQ ID NO:2, a light chain encoded by a nucleic acid having 85%, 90%, 95%, 99%, or 100% sequence identity to SEQ ID NO:5, and a nucleic acid encoding a GPI membrane anchor having 85%, 90%, 95%, 99%, or 100% sequence identity to SEQ ID NO:3. In some embodiments the recombinant genetic construct is an mRNA construct. In some embodiments, the recombinant genetic construct contains signal sequences encoded by a nucleic acid having 85%, 90%, 95%, 99%, or 100% sequence identity to SEQ ID Nos:1 and 4.

One embodiment provides an antibody or antigen fragment thereof having a heavy chain protein sequence having 85%, 90%, 95%, 99%, or 100% sequence identity to SEQ ID NO:7.

One embodiment provides an antibody or antigen binding fragment thereof containing a Decay Accelerating Factor GPI membrane anchor having 85%, 90%, 95%, 99%, or 100% sequence identity to SEQ ID NO:8.

One embodiment provides an antibody or antigen binding fragment thereof containing a light chain signal sequence having 85%, 90%, 95%, 99%, or 100% sequence identity to SEQ ID NO:6.

One embodiment provides an antibody or an antigen binding fragment thereof containing a light chain having 85%, 90%, 95%, 99%, or 100% sequence identity to SEQ ID NO:10.

One embodiment provides an antibody or antigen binding fragment thereof containing a heavy chain having 85%, 90%, 95%, 99%, or 100% sequence identity to SEQ ID NO:7, a GPI membrane anchor having 85%, 90%, 95%, 99%, or 100% sequence identity to SEQ ID NO:8, and a light chain having 85%, 90%, 95%, 99%, or 100% sequence identity to SEQ ID NO:10.

### BRIEF DESCRIPTION OF THE DRAWINGS

FIGS. 1A-1G are PET/CT images of mRNA sprayed onto the cervix and vagina in water using a Teleflex atomizer. The mRNA was labeled with probes from Kirschman et al, NAR, 2017, but with the addition of 64Cu to the streptavidin part of the probe, making them PET active. Longitudinal imaging was performed at 75 min, 4, 24 and 72 hrs demonstrating FRT localization of the mRNA (vagina and cervix) in a macaque.

FIG. 2A is a photograph of aerosol delivery of synthetic mRNA. FIG. 2B is a fluoromicrograph of A549 (lung epithelial) cells treated with 1 μg green fluorescent protein (GFP) encoding synthetic mRNA delivered with aerosol delivery. FIG. 2C is a fluoromicrograph of RAW (macrophage) cells treated with 1 μg green fluorescent protein (GFP) encoding synthetic mRNA delivered with aerosol delivery. FIG. 2D is a fluoromicrograph of normal human bronchial epithelial cells differentiated in an air-liquid interface model cells treated with 1 μg green fluorescent protein (GFP) encoding synthetic mRNA delivered with aerosol delivery. FIG. 2E is a graph of Mander's overlap coefficient versus time showing that when fluorescent probe labeled mRNA were used and colocalizaed with EEA1, CD63 and LAMP1, in A549s, over 75% of the mRNA was cytosolic, indicating non-endosomal delivery.

FIG. 3A is a schematic diagram showing mRNA encoding a secreted IgG PGT121 and a glycosylphosphatidylinositol (GPI)-anchored PGT121, both with NanoLuc® luciferase (NLuc), a 19 kD version of luciferase, fused to the light chain. FIG. 3B is a cartoon depiction of mRNA being sprayed in water and expression and release from epithelial cells; in this case depicting protection from HIV. FIG. 3C is a graph of average radiance (p/s/cm²/sr) for syringe squirt and aerosolized delivery (Teleflex), showing Nanoluc/light chain expression in FRT from mRNA. FIG. 3D is a graph of fold above control for a doses of 250 μg and 750 μg showin that when the dose was increased by 3×, the signal increased. FIG. 3E-3H are fluoromicrographs showing Nanoluc® imaging in the sheep FRT including vagina and cervix 24 hrs post-delivery.

FIGS. 4A-4C are fluoromicrographs showing Nanoluc® signal in the FRT of sheep at 14 (FIG. 4B) and 28 days (FIG. 4C) for the anchored antibody and 14 days for the secreted (FIG. 4A). FIG. 4D is a graph of average radiance (p/s/cm²/sr) for secreted antibody and anchored antibody after 14 days and 28 days. FIG. 4E is a line graph of PGT121 concentration (μg/mL) versus days post transfection for sheep numbers 420, 456, and 461 showing mRNA-encoded antibody expression from the GPI anchored antibody in secretions sampled over 28 days. FIG. 4F is a line graph of PGT121 concentration (μg/mL) versus days post transfection for sheep numbers 414 and 401 showing mRNA-encoded antibody expression in secretions sampled over 21 days. FIG. 4G is a line graph of PGT121 concentration (μg/mL) versus day post transfection showing the mean from FIG. 4E. FIG. 4H is a line graph of PGT121 concentration (μg/mL) versus days post transfection showing the mean of FIG. 44F. FIG. 4I is a micrograph and photograph of a gel showing mRNA-encoded antibody expression from the GPI anchored antibody in cervix, vagina, uterus, and caudal vagina tissue sampled over 28 days.

FIG. 4J is a graph of PGT121 concentration (ng/mg tissue) in cervix, vagina, uterus, and caudal vagina for sheep numbers 456, 420, 461, 452, and 455 at 28 day post transfection.

FIG. 5A is a bar graph of PGT121 concentration (μg/mL) in macaque RVG13 and RWG 13 vaginal secretions showing Expression of mRNA-encoded anchored treated with a low dose (125 ug) of mRNA. FIG. 5B is a line graph of cervical explant SHIV challenge of SIV p27 (pg/mL) versus days of Luciferase negative explants. FIG. 5C is a line graph of cervical explant SHIV challenge of SIV p27 (pg/mL) versus days of Luciferase positive explants. FIG. 5D is a line graph of 50% Neutralization Titers versus Time Post Transfection (hrs.) in Clade B SHIV162p3 for RVg13 (•), RWg13 (▪), and RCo13 (▴). FIG. 5E is a line graph of 50% Neutralization Titers versus Time Post Transfection (hrs.) in Clade B SHIV2873Nip for RVg13 (•), RWg13 (▪), and RCo13 (▴).

### DETAILED DESCRIPTION OF THE INVENTION

### I. Definitions

The term “immunocontraception” does not require that 100% of the subjects receiving the treatment have absolutely no chance of reproducing. Instead, unless denoted otherwise, a subject that has received an immunocontraceptive via gene delivery will have a reduced likelihood of reproducing. In some embodiments, this is reduced by 10, 20, 30, 40, 50, 60, 70, 80, 90, 95, 99, 99.9, 99.99, or 100% (with 100% reduction indicating no chance of reproduction). In some embodiments, the percentage reduced is maintained for at least a satisfactory or desired amount of time. In some embodiments, the reduction is maintained for at least 1 month, for example 2, 3, 4, 5, 6, 7, 8, 9, 10, 11, or 12 months. In some embodiments, the reduction is maintained for at least 1 year, for example, 1, 2, 3, 4, 5, 6, 7, 8, 9, 10, 11, 12, 13, 14, 15, 16, 17, 18, 19, 20, 25, 30, 35, 40, 45, 50, 55, or 60 years. In some embodiments, the reduction is measured and/or set as a fraction of the organism's life, for example, 10, 20, 30, 40, 50, 60, 70, 80, 90, or 100% of the organism's life will be at the noted reduction in likelihood of ability to reproduce. In some embodiments, the reduction is measured and/or set as a fraction of the organism's reproductive life, for example, 10, 20, 30, 40, 50, 60, 70, 80, 90, or 100% of the organism's life will be at the noted reduction in likelihood of ability to reproduce. In some embodiments, the contraceptive can be administered in a single dose, no more frequently than once a year. In some embodiments, the contraceptive can be administered in a single dose, no more frequently than once every 2 years. In some embodiments, the contraceptive can be administered in a single dose, no more frequently than once every 3 years. In some embodiments, the contraceptive can be administered in a single dose, no more frequently than once every 4 years. In some embodiments, the contraceptive can be administered in a single dose, no more frequently than once every 5 years. In some embodiments, the contraceptive can be administered in a single dose, no more frequently than once every 6 years. In some embodiments, the contraceptive can be administered in a single dose, no more frequently than once every 7 years. In some embodiments, the contraceptive can be administered in a single dose, no more frequently than once every 8 years. In some embodiments, the contraceptive can be administered in a single dose, no more frequently than once every 9 years. In some embodiments, the contraceptive can be administered in a single dose, no more frequently than once every 10 years.

A vector that can be used herein includes, but is not limited to, a viral vector, a plasmid, a RNA vector or a linear or circular DNA or RNA molecule which may include a chromosomal, nonchromosomal, semi-synthetic or synthetic DNA. Some vectors are those capable of autonomous replication (episomal vector) and/or expression of nucleic acids to which they are linked (expression vectors). Large numbers of suitable vectors are known to those of skill in the art and commercially available.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)2, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

The term “derivative” refers to an antibody or antigen-binding fragment thereof that immunospecifically binds to the same target of a parent or reference antibody but which differs in amino acid sequence from the parent or reference antibody or antigen binding fragment thereof by including one, two, three, four, five or more amino acid substitutions, additions, deletions or modifications relative to the parent or reference antibody or antigen binding fragment thereof. In some embodiments, such derivatives will have substantially the same immunospecificity and/or characteristics, or the same immunospecificity and characteristics as the parent or reference antibody or antigen binding fragment thereof. The amino acid substitutions or additions of such derivatives can include naturally occurring (i.e., DNA-encoded) or non-naturally occurring amino acid residues. The term “derivative” encompasses, for example, chimeric or humanized variants, as well as variants having altered CH1, hinge, CH2, CH3 or CH4 regions, so as to form, for example antibodies, etc., having variant Fc regions that exhibit enhanced or impaired effector or binding characteristics.

As used herein, a “chimeric antibody” is a molecule in which different portions of the antibody are derived from different immunoglobulin molecules such as antibodies having a variable region derived from a non-human antibody and a human immunoglobulin constant region.

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they should be substantially identical to human immunoglobulin constant regions, i.e., at least about 85-99%, or about 95% or more identical. Hence, all parts of a humanized immunoglobulin, except possibly the CDR's, are substantially identical to corresponding parts of natural human immunoglobulin sequences. A humanized antibody is an antibody including a humanized light chain and a humanized heavy chain immunoglobulin. For example, a humanized antibody would not encompass a typical chimeric antibody, because, e.g., the entire variable region of a chimeric antibody is non-human.

### II. Contraceptive Compositions and Methods

Non-hormonal contraceptive compositions and methods for contraception are provided. One embodiment provides an antibody or an antigen binding fragment thereof that specifically binds to one or more sperm antigens and inhibits the ability of antibody-bound sperm to fertilize an egg. Typically, the antibody is a monoclonal antibody, for example a human or humanized monoclonal antibody. In one embodiment, the antibody or antigen binding fragment thereof specifically binds to CD52g expressed on vertebrate for example human sperm cells and inhibits, blocks, or reduces the ability of the antibody-bound sperm to fertilize an egg. In one embodiment the antibody contains a membrane anchor. The membrane anchor can contain transmembrane domains, glycosylphosphatidylinositol anchors, or myristoylation motifs.

In one embodiment, the complete HCA Heavy Chain mRNA contains a signal sequence, heavy chain sequence, and, if included, membrane anchor sequence. In the following sequences, it will be appreciated that the “T” nucleotides in the following sequences can be replaced with “U” nucleotides to generate similar RNA sequences.

One embodiment provides a vector having a nucleic acid encoding a signal sequence having 85%, 90%, 95%, 99%, or 100% sequence identity to the following sequence: RNA sequence for IgG Heavy Chain Signal Sequence

(SEQ ID NO: 1)
ATGGGCTGGTCCTGCATCATCCTGTTCCTGGTGGCAACCGCAACAGGAG
TGCACAGC.

One embodiment provides an antibody having a heavy chain encoded by a nucleic acid sequence having 85%, 90%, 95%, 99%, or 100% sequence identity to the following sequence:

### RNA Sequence for HCA IgG Heavy Chain

(SEQ ID NO: 2)
CAGGTGCAGCTGCAGCAGTGGGGAGCAGGACTGCTGAAGCCTTCTGAGA
CCCTGAGCCTGACATGTGCCGTGTATGGCGGCAGCTTTTCCGGCTACTA
TTGGTCCTGGATCAGGCAGCCACCTGGCAAGGGACTGGAGTGGATCGGC
GAGATCAACCACTCTGGCAGCACCAACTACAATCCCTCTCTGCGGAGCA
GAGTGACCATCTCCGTGGACACATCTAAGAATCAGTTCTCTCTGAAGCT
GCGCAGCGTGACCGCAGCAGATACAGCCGTGTACTATTGCGCCAGGGGC
TTTATGGTGCGCGGCATCATGTGGAACTACTATTACATGGACGTGTGGG
GCAAGGGCACCACAGTGACCGTGTCCCCATCTGCCAGCACAAAGGGACC
AAGCGTGTTCCCTCTGGCACCAAGCTCCAAGTCCACCTCTGGAGGAACA
GCCGCCCTGGGCTGTCTGGTGAAGGATTATTTCCCTGAGCCAGTGACCG
TGTCCTGGAACTCTGGCGCCCTGACCTCCGGAGTGCACACATTTCCAGC
CGTGCTGCAGTCTAGCGGCCTGTATAGCCTGTCCTCTGTGGTGACCGTG
CCCAGCTCCTCTCTGGGCACCCAGACATACATCTGCAACGTGAATCACA
AGCCAAGCAATACAAAGGTGGACAAGCGGGTGGAGCCCAAGTCCTGTGA
TAAGACCCACACATGCCCACCATGTCCAGCACCTGAGCTGCTGGGAGGA
CCAAGCGTGTTCCTGTTTCCTCCAAAGCCTAAGGACACCCTGATGATCT
CTAGAACCCCCGAGGTGACATGCGTGGTGGTGGACGTGAGCCACGAGGA
TCCTGAGGTGAAGTTCAACTGGTACGTGGATGGCGTGGAGGTGCACAAT
GCCAAGACCAAGCCCCGGGAGGAGCAGTATAACTCCACCTACAGAGTGG
TGTCTGTGCTGACAGTGCTGCACCAGGACTGGCTGAACGGCAAGGAGTA
CAAGTGCAAGGTGTCCAATAAGGCCCTGCCAGCCCCCATCGAGAAGACC
ATCTCTAAGGCAAAGGGACAGCCAAGGGAGCCTCAGGTGTATACACTGC
CCCCTTCCCGCGACGAGCTGACCAAGAACCAGGTGTCTCTGACATGTCT
GGTGAAGGGCTTTTACCCTTCTGATATCGCCGTGGAGTGGGAGAGCAAT
GGCCAGCCAGAGAACAATTATAAGACCACACCACCCGTGCTGGACAGCG
ATGGCTCCTTCTTTCTGTACAGCAAGCTGACCGTGGATAAGTCCCGGTG
GCAGCAGGGCAACGTGTTCAGCTGCTCCGTGATGCACGAGGCCCTGCAC
AATCACTACACCCAGAAGTCTCTGAGCCTGTCCCCTGGCAAG.

One embodiment provides an antibody or an antigen binding fragment thereof containing a GPI membrane anchor encoded by a nucleic acid sequence having 85%, 90%, 95%, 99%, or 100% sequence identity to the following sequence:

### RNA Sequence for Decay Accelerating Factor GPI Membrane Anchor

(SEQ ID NO: 3)
CACGAGACCACACCAAATAAGGGCAGCGGCACCACATCCGGCACCACAA
GACTGCTGAGCGGCCACACCTGTTTTACCCTGACAGGCCTGCTGGGCAC
CCTGGTGACAATGGGCCTGCTGACA.

In one embodiment the complete HCA Light Chain mRNA contains a signal sequence and light chain sequence.

One embodiment provides a vector containing a nucleic acid encoding a signal sequence encoded by a nucleic acid having 85%, 90%, 95%, 99%, or 100% sequence identity to the following sequence:

RNA sequence for IgG Light Chain Signal Sequence

(SEQ ID NO: 4)
ATGGCCTGGACCCCTCTGTGGCTGACACTGTTTACCCTGTGCATCGGCT
CTGTGGTG.

One embodiment provides an antibody or an antigen binding fragment thereof having a light chain encoded by a sequence having 85%, 90%, 95%, 99%, or 100% sequence identity to the following sequence:

RNA sequence for HCA IgG Light Chain

(SEQ ID NO: 5)
AGCTCCGAGCTGACACAGGACCCAGTGGTGAGCGTGGCCCTGGGACAGA
CAGTGCGGATCACCTGTCAGGGCGATTCTCTGAGAACCTACCACGCCAG
CTGGTATCAGCAGAAGCCAAGGCAGGCCCCCGTGCTGGTCATCTACGAC
GAGAACAATAGGCCTTCCGGCATCCCAGATCGCTTCTCCGGCTCTACAA
GCGGCAACACCGCCTCTCTGACAATCACCGGAGCACAGGCAGAGGACGA
GGCAGATTACTATTGCAACTCCCGGGACTCTAGCGGCAATAGACTGGTG
TTCGGAGGAGGAACAAAGCTGACCGTGCTGGGACAGCCAAAGGCAGCAC
CTTCCGTGACCCTGTTTCCACCTTCCTCTGAGGAGCTGCAGGCCAATAA
GGCCACCCTGGTGTGCCTGATCAGCGACTTCTACCCAGGAGCAGTGACA
GTGGCATGGAAGGCCGATAGCTCCCCAGTGAAGGCCGGCGTGGAGACCA
CAACCCCCAGCAAGCAGTCCAACAATAAGTACGCCGCCTCTAGCTATCT
GTCCCTGACCCCCGAGCAGTGGAAGTCTCACAGATCCTATTCTTGCCAG
GTGACACACGAGGGCAGCACAGTGGAGAAGACCGTGGCCCCTACAGAGT
GTTCC.

One embodiment provides an antibody or antigen fragment thereof having a heavy chain encoded by a nucleic acid sequence having 85%, 90%, 95%, 99%, or 100% sequence identity SEQ ID NO:2, a GPI membrane anchor encoded by a nucleic acid sequence having 85%, 90%, 95%, 99%, or 100% sequence identity to SEQ ID NO:3, and a light chain encoded by a sequence having 85%, 90%, 95%, 99%, or 100% sequence identity to SEQ ID NO:5.

One embodiment provides an antibody or antigen fragment thereof having a heavy chain signal sequence having 85%, 90%, 95%, 99%, or 100% sequence identity to MGWSCIILFLVATATGVHS (SEQ ID NO:6).

One embodiment provides an antibody or antigen fragment thereof having a heavy chain protein sequence having 85%, 90%, 95%, 99%, or 100% sequence identity to:

(SEQ ID NO: 7)
QVQLQQWGAGLLKPSETLSLTCAVYGGSFSGYYWSWIRQPPGKGLEWIG
EINHSGSTNYNPSLRSRVTISVDTSKNQFSLKLRSVTAADTAVYYCARG
FMVRGIMWNYYYMDVWGKGTTVTVSPSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGK.

One embodiment provides an antibody or antigen binding fragment thereof containing a Decay Accelerating Factor GPI membrane anchor having 85%, 90%, 95%, 99%, or 100% sequence identity to:

(SEQ ID NO: 8)
HETTPNKGSGTTSGTTRLLSGHTCFTLTGLLGTLVTMGLLT.

One embodiment provides an antibody or antigen binding fragment thereof containing a light chain signal sequence having 85%, 90%, 95%, 99%, or 100% sequence identity to:

(SEQ ID NO: 9)
MAWTPLWLTLFTLCIGSVV.

One embodiment provides an antibody or an antigen binding fragment thereof containing a light chain having 85%, 90%, 95%, 99%, or 100% sequence identity to:

(SEQ ID NO: 10)
SSELTQDPVVSVALGQTVRITCQGDSLRTYHASWYQQKPRQAPVLVIY
DENNRPSGIPDRFSGSTSGNTASLTITGAQAEDEADYYCNSRDSSGNR
LVFGGGTKLTVLGQPKAAPSVTLFPPSSEELQANKATLVCLISDFYPG
AVTVAWKADSSPVKAGVETTTPSKQSNNKYAASSYLSLTPEQWKSHRS
YSCQVTHEGSTVEKTVAPTECS.

One embodiment provides an antibody or antigen binding fragment thereof containing a heavy chain having 85%, 90%, 95%, 99%, or 100% sequence identity to SEQ ID NO:7, a GPI membrane anchor having 85%, 90%, 95%, 99%, or 100% sequence identity to SEQ ID NO:8, and a light chain having 85%, 90%, 95%, 99%, or 100% sequence identity to SEQ ID NO:10.

Another embodiment provides a recombinant genetic construct. The construct encodes an antibody or antigen binding fragment thereof that specifically binds to a sperm antigen and a membrane anchor. The genetic construct can be configured to be delivered and expressed in an animal subject, for example a human. In one embodiment, the recombinant genetic vector includes a nucleic acid encoding a heavy chain encoded by a nucleic acid having 85%, 90%, 95%, 99%, or 100% sequence identity to SEQ ID NO:2, a light chain encoded by a nucleic acid having 85%, 90%, 95%, 99%, or 100% sequence identity to SEQ ID NO:5, and a nucleic acid encoding a GPI membrane anchor having 85%, 90%, 95%, 99%, or 100% sequence identity to SEQ ID NO:3. In some embodiments the recombinant genetic construct is an mRNA construct. In some embodiments, the recombinant genetic construct contains signal sequences encoded by a nucleic acid having 85%, 90%, 95%, 99%, or 100% sequence identity to SEQ ID Nos:1 and 4.

Another embodiment provides a therapeutic mRNA that expresses an antibody or antigen binding fragment there that specifically binds to sperm and inhibits antibody-bound sperm for fertilizing an egg. In some embodiments, the antibody is an immunoglobulin G, immunoglobulin M, immunoglobulin A, immunoglobulin D, or immunoglobulin E. In one embodiment the antibody specifically binds to CD52g expressed on sperm cells. In some embodiments the antibody contains a membrane anchor. The membrane anchor can contain transmembrane domains, glycosylphosphatidylinositol anchors, or myristoylation motifs.

Another embodiment provides a pharmaceutical composition containing a nucleic acid construct encoding an antibody or antigen binding fragment thereof that specifically binds to a sperm antigen and a membrane anchor. In one embodiment the nucleic construct is an mRNA construct, for example an mRNA construct. In one embodiment the sperm antigen is CD25g. In some embodiments, the pharmaceutical composition contains an excipient. In some embodiments, the excipient is water. The membrane anchor can contain transmembrane domains, glycosylphosphatidylinositol anchors, or myristoylation motifs.

One embodiment provides a kit containing a nucleic acid construct encoding an antibody or an antigen-binding fragment thereof that specifically binds to a sperm antigen and also encodes a membrane anchor, and a delivery device. In some embodiments the delivery device is and atomizer or a dual-chamber syringe containing lyophilized mRNA and water (allowing for cold-chain independence), and an atomizer suitable for self-insertion into the FRT. Exemplary atomizers that can be used to deliver mRNA encoding anti-CD52g antibodies include but are not limited to a Penn Century microsprayer (20 um), Teleflex atomizer (30-100 um), an impinging jet atomizer (5-10 um), a pediatric nebulizer (5-7 um), and a droplet stream generator. The atomizers can be used to vary both droplet velocity and size. The items of the kit are within a container. The container can also include written instructions for using the kit. In one embodiment, the mRNA encoding anti-CD52g antibodies are delivered to the FRT with a dual-chamber syringe containing lyophilized mRNA and water (allowing for cold-chain independence), and an atomizer suitable for self-insertion into the FRT.

One embodiment provides a pharmaceutical composition consisting of an mRNA vector or construct encoding an antibody or antibody-binding fragment thereof that specifically binds to sperm antigen and inhibits or blocks antibody-bound sperm from fertilizing an egg and water. The discovery that synthetic mRNA in water can be delivered to the female reproductive tract (FRT) mucosal surfaces via aerosol may have far reaching consequences for contraception and female reproductive health. One embodiment provides mRNA encoded antibodies the specifically bind to CD52g expressed on sperm cells. In one embodiment, the mRNA encoded antibodies are delivered to the FRT using a microsprayer or atomizer.

In one embodiment the antibody or antigen binding fragment thereof specifically binds to CD52g expressed on sperm cells. In other embodiments, the antibody or antigen binding fragment thereof specifically binds to sperm adhesion molecule 1 (SPAM 1), metalloprotease disintegrin cysteine (MDC), sperm protein (SP-10), fertilization antigen (FA-1), SP-17, NZ-1, NZ-2, lactate dehydrogenase (LDH-C4), sperm agglutination antigen (SAGA-1), YLP-12 peptide, human equatorial segment protein (hESP), BS-17, rabbit sperm membrane protein-B (rSMP-B), sperm acrosomal membrane-associated protein (SAMP-32), and 80 kDa human sperm antigen (HSA). In other embodiments, the antibody or antigen-binding fragment thereof binds to dorsal head and equatorial (DE), epididymal protease inhibitor (Eppin), and sperm flagella protein (SFP-2) (Kiranjeet Kaur, Vijay Prabha, “Immunocontraceptives: New Approaches to Fertility Control”, BioMed Research International, vol. 2014, Article ID 868196, 15 pages, 2014). The amino acid sequences of the listed sperm antigens are known in the art.

Exemplary antibodies that can be used for contraception include antibodies disclosed in US Patent Application Publication 20140223591 which is incorporated by reference in its entirety or P. E. Castle, K. J. Whaley, T. E. Hoen, T. R. Moench, and R. A. Cone, “Contraceptive effect of sperm-agglutinating monoclonal antibodies in rabbits,” Biology of Reproduction, vol. 56, no. 1, pp. 153-159, 1997, which is also incorporated by reference in its entirety. It will be appreciated that these antibodies can be humanized and modified to include a membrane anchor.

It has been discovered that mRNA delivered via aerosol can express sufficient quantities of protein to achieve therapeutic and/or preventive efficacy at a mucosal site. We extended this approach to the FRT of sheep and macaques, as shown in our preliminary data, using an off-the shelf Teleflex atomizer for delivery. In some embodiments, the cells in the vagina and/or the cervix are transfected with mRNA encoding anti-CD52g antibodies suspended in water and delivered with an atomizer. The discovery that synthetic mRNA in water can be delivered to mucosal surfaces of the FRT via aerosol introduces the possibility that contraceptive proteins such as antisperm Abs may be delivered by this mechanism, as well as products that may have other beneficial effects on reproductive health such as Abs that specifically bind to sexually transmitted organisms, antimicrobial peptides and antigens for elicitation of local immune responses.

Anti-sperm Abs commonly occur in infertility patients, and are thought to cause infertility due to sperm agglutination and immobilization (Bronson, RA., J. Reprod. Immunol., 45(2):159-83 (1999); Ustay, K, et al., Univ Mich Med Cent J., 33(5):225-7 (1967)). Abs found in some immune infertile patients are directed against a glycoprotein called CD52g, a molecule unique to the male reproductive tract and initially detected on the surface of sperm. CD52g is related to CD52, a molecule expressed by T lymphocytes, but differs in its carbohydrate side chain that contains the epitope that is specific for the male reproductive tract. CD52g is produced and secreted by epithelial cells lining the lumen of the epididymis, vas deferens, and seminal vesicles (Norton, E J., et al., Tissue Antigens, 60(5):3 (2002)). It contains a glycosylphosphatidylinositol (GPI) anchor, and is transferred to the plasma membrane of sperm as they mature in the epididymis (Diekman, A B., et al., Immunol., Rev., 171:203-11(1999); Diekman, A B, et al., Am. J. Reprod. Immunol., 43(3):134-43 (2000)). Isojima and coworkers made two monoclonal Abs against CD52g: HC4, a human IgM Ab made from B cells of an infertile woman, and 2C6, a mouse monoclonal Ab with the same specificity (Isojima, S., et al., J. Reprod. Immunol., 10(1):67-78 (1987)). A WHO-sponsored contraceptive vaccine workshop that examined the function and specificity of these and other antisperm monoclonal Abs identified CD52g as a promising anti-fertility vaccine candidate due to its unique expression in the male reproductive tract, potent antigenicity, and its ability to induce infertility in otherwise healthy individuals (Anderson, D J, et al., J. Reprod. Immunol., 10(3):231-57(1987)). While systemic Abs have multiple potential effector functions (e.g. complement dependent cytotoxicity (CDC), Ab dependent cellular cytotoxicity (ADCC)), there are also mucosal-specific mechanisms including agglutination (Cone, R A, et al., Am. J. Reprod. Immunol., September; 32(2):114-31 (1994); Roche, A M, et al., Mucosal Immunology., 8(1):176— 85 (2015)) and binding to mucus (Phalipon, A, et al., Immunity., 17(1):107-15 (2002); Wang, Y-Y, et al., Eur. Respir. J., 49(1):1601709 (2017)) that are less widely discussed, but are crucial for the protection of mucosal surfaces. These effector functions for Abs in mucus serve to block the movement of entities such as viruses, bacteria, infected cells, and sperm, and prevent them from reaching target cells. Drs. Anderson, Whaley and Moench have produced a human anti-CD52g Ab in Nicotiana based on the sequence of the original HC4 Ab, and call this Ab “Human Contraceptive Antibody” (HCA). Their studies have shown that HCA, like the parent Ab, potently agglutinates sperm and immobilizes sperm in cervicovaginal mucus.

In one embodiment the mRNA encoding the anti-CD52g antibodies are produced by large-scale production of under GMP conditions is easy, robust, and inexpensive, when compared to the production of peptides, proteins, modified microorganisms and cells (Tusup, M, and Pascolo S., Methods Mol. Biol. New York, N.Y.: Springer New York, 1499 (Chapter 9):155-63 (2017)). In one embodiment the transcription reaction produces the same final concentration of mRNA whether it is performed in a 10 ul or 10 ml. Upscaling the transcription reaction to a volume of a liter or more should not pose any problems. Using established conditions to produce a GMP molecule with a different sequence. Every mRNA molecule consists of A, C, G and U residues. Thus, the final molecule will always be soluble and stable at neutral pH and will not present any unpredictable behavior. Accordingly, established methods can be used for the production of any mRNA under GMP conditions. Lyophilization/resolubilization: mRNA can be lyophilized and resuspended immediately in water-based solutions regardless of its sequence. Storage and temperature stability: mRNA in solution can be stored for weeks at room temperature as long as it is pure and in a neutral or acidic solution. Lyophilized mRNA can be stored for months at room temperature.

Synthetic mRNA has a number of properties ideal for in vivo expression of Abs as compared with viral vectors and DNA. The expression is transient compared with viral vectors, and the RNA is non-integrating. mRNA stability in vivo is controllable, to some degree, via the UTR sequences, and mRNA will always degrade. Therefore Ab production will not be permanent, an important feature for human immunocontraception. The transient nature, though, does not preclude the ability to produce a durable Ab presence in vaginal secretions. Sheep data shows high levels of mRNA-expressed Ab in sheep secretions for 20 days following a single administration of mRNA encoding simple (unlinked) Ab, and 28 days following a single administration of mRNA encoding a GPI-linked Ab. In addition, synthetic mRNA has not been observed in the nucleus, and it is unlikely to be integrated. DNA must reach the nucleus to function and thus can interact with chromatin; this is not the case with mRNA.

The mRNA does not provoke a significant immune response. Two approaches were used for mitigating innate immune responses to the RNA itself: First, the mRNA was modified with N1-methyl-pseudouridine, and reverse phase HPLC was used to reduce double-stranded aberrant RNAs, etc. It was recently reported that cytokines were not elevated in the mouse lung after mRNA delivery (Tiwari, P M, et al., Nature Communications., 9(1):3999 (2018)).

mRNA can transfect difficult to transfect cell types. Given that the mRNA is delivered to the cytosol, many cells that are difficult to transfect with DNA can be transfected with mRNA.

The ability to express Abs in the FRT using such a simple formulation via clearly separates mRNA from DNA delivery which is usually achieved by injection or via the use of viral vectors. In some embodiments a Penn Century microsprayer or a Teleflex MADgic Laryngo-Tracheal Mucosal Atomization Device can be used to transfect tissue culture cells in dishes, mouse lung epithelial cells in vivo, and the vagina and cervix of sheep and macaques in vivo, using mRNA in water. As little as 125 ug of mRNA in sheep has been used to transfect the cervix, and 100 ug to transfect mouse lungs.

A. Pharmaceutical Compositions

Pharmaceutical compositions including the disclosed nucleic acid constructs are provided. Pharmaceutical compositions containing the nucleic acid construct can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed nucleic acid constructs, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed nucleic acid constructs, generally dosage levels of 0.001 to 20 mg/kg of body weight daily are administered to mammals. Generally, for intravenous injection or infusion, dosage may be lower.

In certain embodiments, the nucleic acid constructis administered locally, for example by injection directly into a site to be treated. Typically, the injection causes an increased localized concentration of the nucleic acid constructcomposition which is greater than that which can be achieved by systemic administration. The nucleic acid constructcompositions can be combined with a matrix as described above to assist in creating an increased localized concentration of the polypeptide compositions by reducing the passive diffusion of the polypeptides out of the site to be treated.

1. Formulations for Parenteral Administration

In some embodiments, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Formulations for Topical Administration

The disclosed nucleic constructs can be applied topically. Topical administration does not work well for most peptide formulations, although it can be effective especially if applied to the lungs, nasal, oral (sublingual, buccal), vaginal, or rectal mucosa.

Formulations for administration to the mucosa will typically be spray dried drug particles, which may be incorporated into a tablet, gel, capsule, suspension or emulsion. Standard pharmaceutical excipients are available from any formulator.

Transdermal formulations may also be prepared. These will typically be ointments, lotions, sprays, or patches, all of which can be prepared using standard technology. Transdermal formulations may require the inclusion of penetration enhancers.

3. Controlled Delivery Polymeric Matrices

The nucleic constructs disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of nucleic acids constructs, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

B. Methods of Use

One embodiment provides a method for providing contraception to a female subject in need thereof including the steps of administering to the subject's female reproductive tract a nucleic acid construct encoding an antibody or an antigen binding fragment thereof and a membrane anchor in an amount effective to provide contraception. In one embodiment, the nucleic acid construct is an mRNA construct. In some embodiments the construct is delivered as an aerosol. In other embodiments, the construct is delivered using nanoparticles, for example lipid nanoparticles containing polyethylenimine (PEI) or modified PEI. In some embodiments the construct can be delivered using poly-beta-amino-esters nano-vehicles (PBAEs), and modified PBAEs.

A typical subject is a human, fertile, female. An effective amount of a nucleic acid construct encoding an antibody or an antigen-binding fragment thereof is delivered to the reproductive tract of the subject to provide contraception. The construct transfects cells in the female reproductive tract, for example vaginal and cervical epithelial cells and is expressed. The expressed antibody then binds to sperm in the reproductive tract. The antibody-bound sperm cannot bind to and fertilize an egg. In some embodiment the antibody binds to CD52g expressed on sperm. In other embodiments, the antibody specifically binds to to sperm adhesion molecule 1 (SPAM 1), metalloprotease disintegrin cysteine (MDC), sperm protein (SP-10), fertilization antigen (FA-1), SP-17, NZ-1, NZ-2, lactate dehydrogenase (LDH-C4), sperm agglutination antigen (SAGA-1), YLP-12 peptide, human equatorial segment protein (hESP), BS-17, rabbit sperm membrane protein-B (rSMP-B), sperm acrosomal membrane-associated protein (SAMP-32), and 80 kDa human sperm antigen (HSA). In other embodiments, the antibody or antigen-binding fragment thereof binds to dorsal head and equatorial (DE), epididymal protease inhibitor (Eppin), and sperm flagella protein (SFP-2) (Kiranjeet Kaur, Vijay Prabha, “Immunocontraceptives: New Approaches to Fertility Control”, BioMed Research International, vol. 2014, Article ID 868196, 15 pages, 2014).

Another embodiment provides a method for providing contraception to a female subject in need thereof by transfecting FRT epithelial cells with a nucleic acid construct encoding an antibody or an antigen-binding fragment thereof that specifically binds to a sperm antigen and also encodes a membrane anchor in an amount effective to provide contraception. \

### III. Methods of Manufacture

A. Methods of Making Antibodies

The disclosed anti-sperm antigen antibodies can be generated in cell culture, in phage, or in various animals, including but not limited to cows, rabbits, goats, mice, rats, hamsters, guinea pigs, sheep, dogs, cats, monkeys, chimpanzees, and apes. Therefore, in one embodiment, an antibody is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); Current Protocols In Immunology (John Wiley & Sons, most recent edition).

The disclosed anti-sperm antigen antibodies can be modified by recombinant means to increase greater efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol. Immunol. 30:105-08 (1993). The modification in amino acids includes deletions, additions, and substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to proteins, polypeptides, or fusion proteins of FLRT3. See, e.g., Antibody Engineering: A Practical Approach (Oxford University Press, 1996).

For example, suitable antibodies with the desired biologic activities can be identified using in vitro assays including but not limited to: proliferation, migration, adhesion, soft agar growth, angiogenesis, cell-cell communication, apoptosis, transport, signal transduction, and in vivo assays such as the inhibition of tumor growth. The antibodies provided herein can also be useful in diagnostic applications. As capture or non-neutralizing antibodies, they can be screened for the ability to bind to the specific antigen without inhibiting the receptor-binding or biological activity of the antigen. As neutralizing antibodies, the antibodies can be useful in competitive binding assays.

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the non-modified antibody or antibody fragment.

Techniques can also be adapted for the production of single-chain antibodies specific to an antigenic peptide. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

A monoclonal antibody is obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

Monoclonal antibodies can be made using any procedure which produces monoclonal antibodies. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

Antibodies may also be made by recombinant DNA methods. DNA encoding the disclosed antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

Methods of making antibodies using protein chemistry are also known in the art. One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. Alternatively, the peptide or polypeptide is independently synthesized in vivo as described above. Once isolated, these independent peptides or polypeptides may be linked to form an antibody or antigen binding fragment thereof via similar peptide condensation reactions.

For example, enzymatic ligation of cloned or synthetic peptide segments allow relatively short peptide fragments to be joined to produce larger peptide fragments, polypeptides or whole protein domains. Alternatively, native chemical ligation of synthetic peptides can be utilized to synthetically construct large peptides or polypeptides from shorter peptide fragments. This method consists of a two-step chemical reaction. The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site.

B. Methods for Producing Isolated Nucleic Acid Molecules Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Protein-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992.

### EXAMPLES

### Example I: Use of Membrane Linkers as a Controlled Release Mechanism

Given the transient nature of mRNA expression, the pharmacokinetics of Ab production and secretion were controlled by engineering the Ab. It has been demonstrated in the mouse lung and sheep FRT that through the incorporation of a GPI-linker from decay accelerating factor (DAF) into the Ab heavy chain, Ab could be retained in the tissues, and detect high concentrations of Ab in secretions for over 28 days following a single administration. By varying the linker design, the tissue and secretion pharmacokinetics van be “tuned” to the temporal window of contraception. In addition, a by-product of the use of the linker, was the observation that mRNA-expressed Abs traffic through the ER and Golgi more efficiently, promoting antibody production, membrane display, and release from the membrane. Most GPI linked proteins traffic to the apical membrane of polarized epithelial cells. This is clearly beneficial for exogenously expressed antibodies. True Ab secreting cells (ASC) are specialized for secreting antibody and expand their ER via IRE1-XBP1 pathway activation. This pathway is not often activated in epithelial cells, and thus these cells do not typically have the capacity to move antibody efficiently through the ER and Golgi. pERp1 and BiP are also important for Ab assembly and not often expressed outside of lymphocytes. It was found that by incorporating linkers into the heavy chain, ER trafficking of Abs produced in non-ASC cells without the benefit of these proteins and pathways was improved. Co-expression of XBP1, pERp1 and BiP, can be explored as a means of improving Ab production.

DAF GPI linkers are well-studied and have demonstrated varying susceptibility to cleavage from phospholipase D and C (PLD and PLC) (Davitz, M A., J Exp Med., 1; 163(5):1150-61(1986); bovine AChE has been shown to be more resistant to PLDD, while susceptible to PLC, and human AchE is highly resistant to PLC and PLD. The resistance is due to the existence of an additional fatty acid chain on the inositol ring which blocks the action of PLC. In addition to GPI linkers, a transmembrane domain™ from MUC4, a typically expressed, cell-surface, mucin can be used, and a fusion between the MUC4 TM domain and peptides identified as substrates for kallikrein-related peptidase 13 (KLK13) (Muytjens, C M J, et al., Nature Publishing Group, 7; 13(10):596-607 (2016); Shaw, J L V, et al., Biological Chemistry., 389(12):561-10 (2008); Andrade, D., et al. Biochimie. Elsevier Masson SAS, 93(10):1701-9 2011)), both active in the FRT. Through the use of these various linkers, tissue retention and release rate of Ab from the cell surface, and into the cervicovaginal fluid can be controlled.

### Example II: Use of Imaging Tools to Assess mRNA Delivery at the Whole Body to Single Cell Level

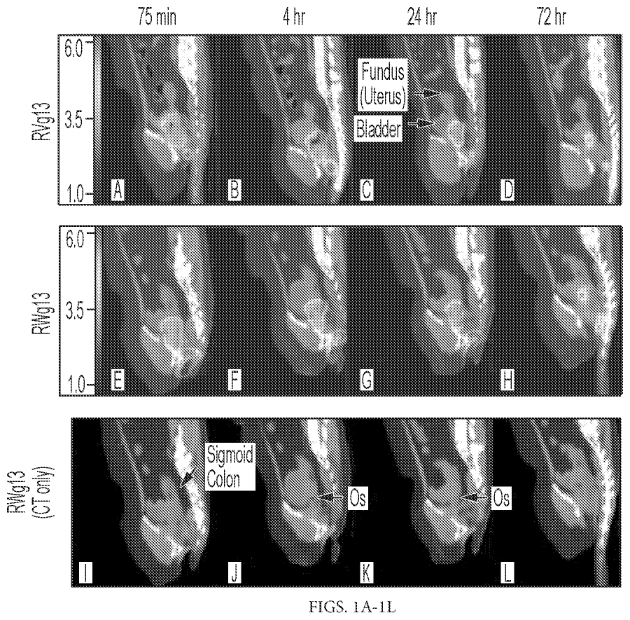

RNA imaging probes (Kirschman, J L, et al., Nucleic Acids Res., 45(12):e113-3 (2017)) PMCID: PMC5499550) can be used in the 3′-UTR of the mRNA, that neither interfere with translation of mRNA nor induce innate immune responses. These probes allow for fluorescence microscopy with single RNA sensitivity, near-IR imaging using a hand-held imaging device and “IVIS” imaging, as well as compatibility with radionuclides for PET imaging through the labeling of the streptravidin with DOTA/64Cu (Santangelo, P J, et al., Mucosal Immunology. Society for Mucosal Immunology, December 20; 107:53 (2017); Santangelo, P J, et al., Nat. Methods. Nature Publishing Group; May; 12(5):427-32. PMCID: PMC4425449(2015)) (FIGS. 1A-1L).

The ability to localize delivered mRNAs both using fluorescence colocalization analysis and can be done using proximity ligation assays (PLA) in multiple cell types. PLA only yields a fluorescent puncta when the mRNA and protein of interest (e.g., endocytic markers) are within ˜30 nm of each other, where typical colocalization analysis is limited by the diffraction limit. PLA allows for the quantification of RNA-protein interactions over many cells and can identify mRNA entry pathways (42). Colocalization analysis is often used as an initial screen, followed by PLA, and then super-resolution microscopy (dSTORM) to confirm the findings (4, 49, 50). dSTORM is a subdiffraction-limited imaging approach with 20-30 nm resolution. Electron microscopy can also be used to determine if transient pores are developed during delivery, in conjunction with the Emory Electron Microscopy Core. mRNAs. Synthetic mRNAs encoding a (DAF) GPI-anchored nanoluc can be used. A single reporter mRNA will allow for both optimization of atomization parameters and for the interrogation of the mechanism of delivery. Anchored nanoluc allows for both nanoluc-assays to be performed, which are quantitative, and immunostaining for tissue and cellular localization. Immunostaining for endocytic markers. Reagents for clathrin light chain and heavy chain, caveolin 1 and 3, EEA1, Rab5, Rab7, CD63, and LAMP-1, for mouse (42,48), human and monkey species have been validated. Use of endocytosis inhibitors. Methyl-3-cyclodextrin (M3CD) and chlorpromazine inhibit clathrin mediated endocytosis, while Filipin III can be used to inhibit cavaelae mediated endocytosis. Blebbistatin is a general inhibitor of myosin II, ATP depletion and 4C are more general inhibitors of endocytosis. All of the drugs can be titrated in conjunction with fluorescently labeled dextran to verify function. ATP depletion is achieved via incubation with glucose-free medium or Ringers buffer containing antimycin A, 2-deoxy-D-glucose (2DG) and sodium azide (NaN3). Concentrations can be optimized for the vaginal and endocervical cultures. Use of in vitro models to study effects of hormones. The Anderson laboratory has shown that the EpiVaginal model expresses hormone receptors and mounts characteristic responses when treated with estrogen and progesterone at concentrations similar to those found during the proliferative and luteal phases of the menstrual cycle (36). EpiVaginal tissues can be pretreated with hormone combinations to determine whether hormone status affects mRNA transfection and translation. Induction of tissue lesions, inflammation. Microabrasions can be introduced in the Epivaginal model by repeated taping of the tissue with a fine-gauge needle. To simulate inflammation, tissue can be treated with 25 ug of TNF-α which causes a breakdown of apical surface tight junctions.

FIGS. 1A-1G are PET/CT images of mRNA sprayed onto the cervix and vagina in water using a Teleflex atomizer. The mRNA was labeled with probes from Kirschman et al, NAR, 2017, but with the addition of 64Cu to the streptavidin part of the probe, making them PET active. Longitudinal imaging was performed at 75 min, 4, 24 and 72 hrs demonstrating FRT localization of the mRNA (vagina and cervix) in a macaque.

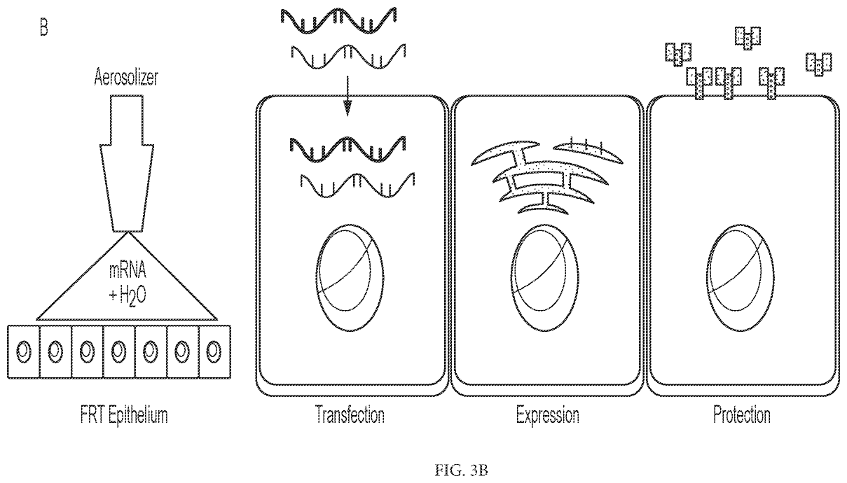

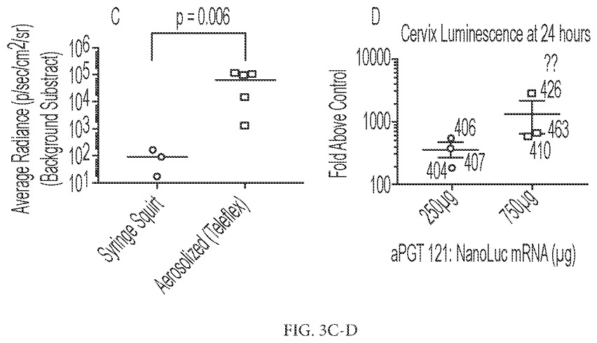

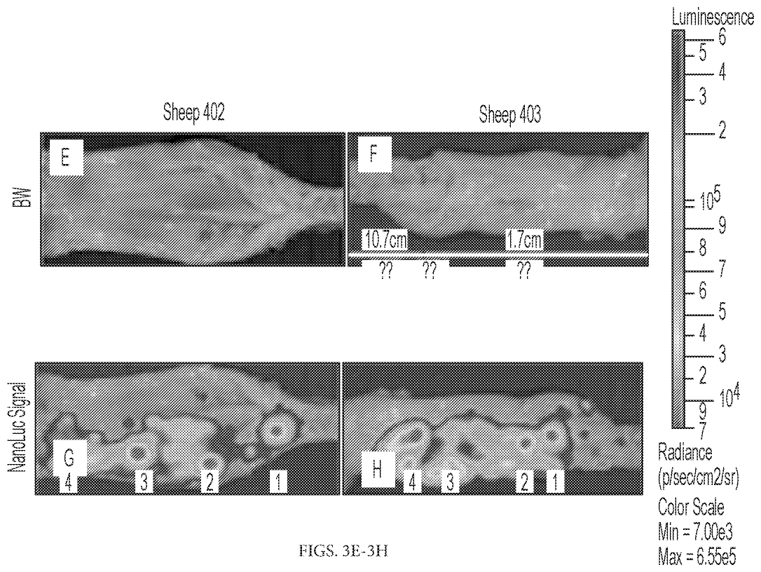

It was demonstrated that the light chain of the Ab fused to a “nanoluc” (19 kD version of luciferase) reporter (FIGS. 3E-H). FIG. 3E-3H are fluoromicrographs showing Nanoluc imaging in the sheep FRT including vagina and cervix 24 hrs post-delivery. This has enabled the direct visualization of Ab expression in cervical and vaginal tissues dissected from sheep as it is ATP independent and functions attached to the cell surface. This is an invaluable tool for imaging expression at the whole tissue level in the FRT.

### Example III: Delivery and Expression of Ab mRNA in FRT Tissues

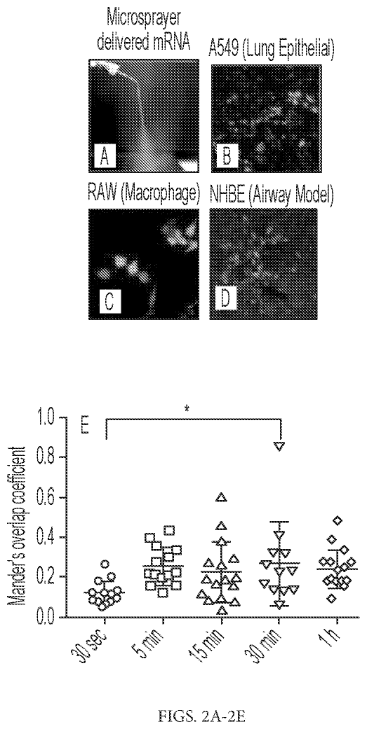

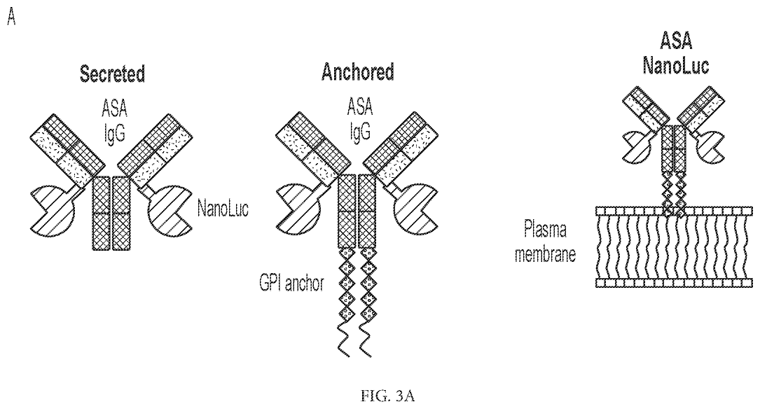

mRNA delivery at mucosal sites using mRNA formulated in water and delivered via aerosol was performed using the Penn-Century microsprayer which produces 20 um droplets, and a Teleflex Madgic atomizer, which produces 30-100 um sized droplets, for in vitro (FIGS. 2A-2E) and in vivo delivery (FIGS. 3A-3H, 4A-4J, and 5A-5E) of mRNA. When mRNA were added dropwise in water, in vitro, or with a syringe (jet, no atomization) in vivo, transfection did not occur, demonstrating the need for atomization. To date, water has been the only fluid used, based on literature suggesting that hypotonic solutions would augment delivery. Hypotonic solutions, such as water, when delivered via aerosol, alter the pressure at the membrane facilitating pore formation and direct access of the mRNA to the cytosol. In water, the mRNA will also have a highly reduced secondary structure, which may also facilitate entry. It was found in an earlier study using AAV-vectored Ab DNA, that vaginal tissue was relatively resistant to gene delivery unless microlesions were introduced which allowed the vectored DNA to reach the basal epithelial cell layer; based on this result, AAV-vectored minibodies were delivered to the macaque FRT by inducing mild abrasion with a cytobrush. In one embodiment, delivery of the mRNA to the FRT includes gentle abrasion to greatly enhance Ab expression from mRNA.

### Example IV: In Vitro Delivery of mRNA Via Aerosol

Apenn-Century microsprayer was used to deliver synthetic mRNA encoding GFP to A549 cells, RAW 264.7 macrophages, and polarized, ciliated lung epithelial cells. The polarized epithelial cells were ciliated and contained mucus producing goblet cells. In each case, when 1000 ng of mRNA in water were sprayed on the cells using 50 ul volumes, over 70% of the cells were transfected near the center of the spray area (FIGS. 2A-2E). In addition, when the mRNA were fluorescently labeled as per Kirschman et al., delivered via microsprayer to A549 cells, and immunostained for EEA1, CD63 and LAMP1, over 80% of the mRNA was cytosolic at 30 s, and over 75% was cytosolic after 1 hr (FIG. 2E). This data suggests that the delivery is direct to the cytosol.

### Example IV: FRT Delivery of mRNA Encoded Antibodies Via Aerosol

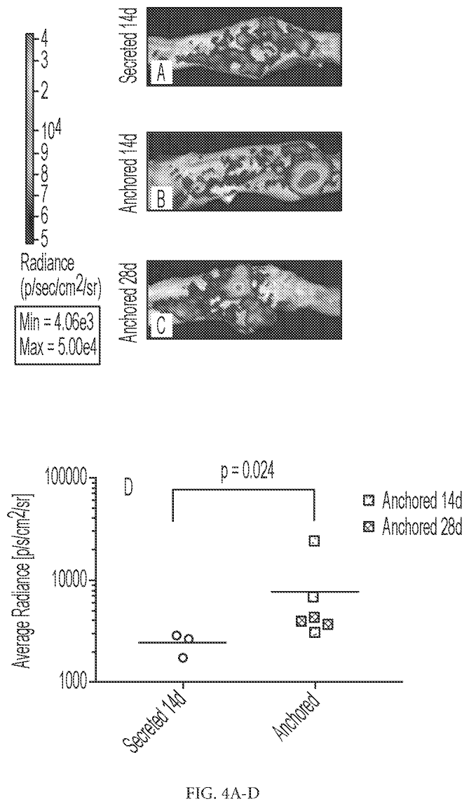

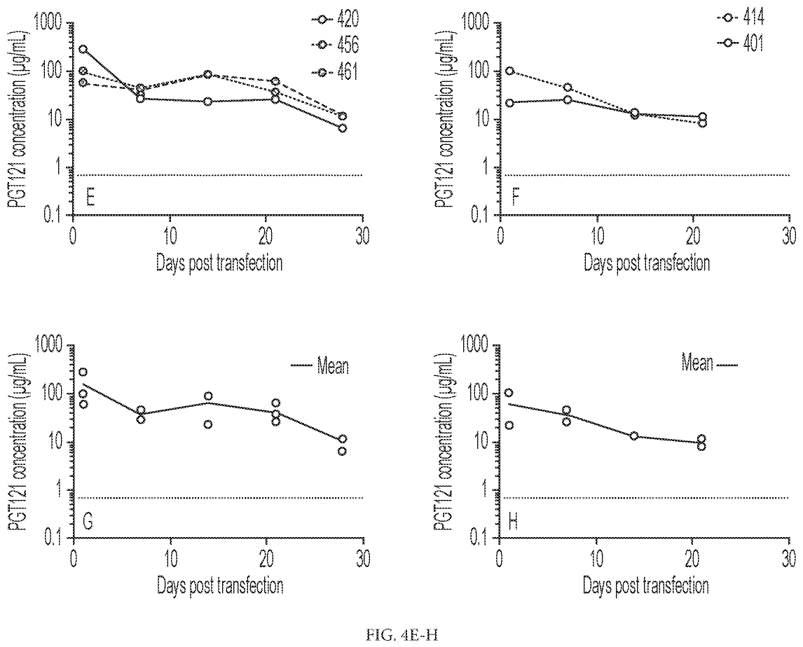

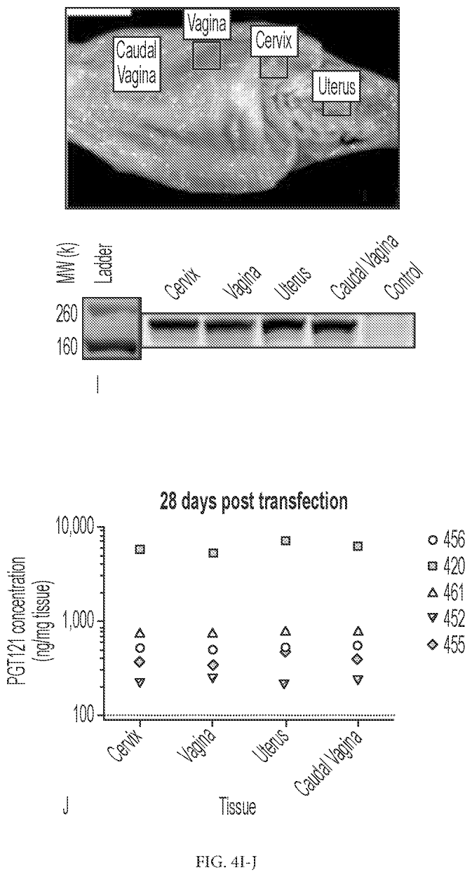

A mAb (IgG) against HIV, PGT121 with and without the GPI anchor. Nanoluc® was added a to the light chain to visualize expression in relevant tissues was expressed in the FRT of sheep. In FIGS. 3A and 3B show a schematic of the Abs used and the general concept. In FIGS. 4C and 4D show that aerosolization was required for expression, as a syringe “squirt” of the mRNA did not result in nanoluc production, and that by increasing the dose, an increase expression was observed. Next, at 24 hrs post-delivery shown in FIGS. 4E-4H, demonstrate that the cervix cells and vagina cells are all capable of transfection. One third of the total RNA dose (750 ug) was delivered to the cervix, and the other two-thirds to the vagina. A more even distribution within the vagina can be achieved through atomizer design.

### Example V: In Vitro Models of the Vagina and Cervix

3D models of human vaginal and endocervical epithelia can be used. These models, which are comprised of a differentiated epithelium on a fibroblast-containing matrix, and morphologically and functionally resemble the tissue of origin, are highly reproducible and remain viable for 10+days after differentiation. Ex vivo FRT tract tissues collected from women at the time of hysterectomy of vaginal repair surgery can also be used. Advantages of ex vivo tissues are the presence of immune cells (dendritic cells, lymphocytes, macrophages), which is important for determining whether immune cells incorporate and express exogenous RNA. However these tissues do not remain intact for long (<24 hours) and there is a high degree of variablility between donors.

The rhesus macaque model can be used for experiments monitoring long term expression of Abs. They are more compatible than sheep or mice regarding the use of human Abs and human sperm.

### Example VI: Expression of mRNA-Encoded Antibodies for Over 28 Days in the Sheep Model

FIGS. 4A-4C are fluoromicrographs showing Nanoluc® signal in the FRT of sheep at 14 (FIG. 4B) and 28 days (FIG. 4C) for the anchored antibody and 14 days for the secreted (FIG. 4A). FIG. 4D is a graph of average radiance (p/s/cm²/sr) for secreted antibody and anchored antibody after 14 days and 28 days. FIG. 4E is a line graph of PGT121 concentration (μg/mL) versus days post transfection for sheep numbers 420, 456, and 461 showing mRNA-encoded antibody expression from the GPI anchored antibody in secretions sampled over 28 days. FIG. 4F is a line graph of PGT121 concentration (μg/mL) versus days post transfection for sheep numbers 414 and 401 showing mRNA-encoded antibody expression in secretions sampled over 21 days. FIG. 4G is a line graph of PGT121 concentration (μg/mL) versus day post transfection showing the mean from FIG. 4E. FIG. 4H is a line graph of PGT121 concentration (μg/mL) versus days post transfection showing the mean of FIG. 44F. FIG. 4I is a micrograph and photograph of a gel showing mRNA-encoded antibody expression from the GPI anchored antibody in cervix, vagina, uterus, and caudal vagina tissue sampled over 28 days. FIG. 4J is a graph of PGT121 concentration (ng/mg tissue) in cervix, vagina, uterus, and caudal vagina for sheep numbers 456, 420, 461, 452, and 455 at 28 day post transfection.

### Example VII: Macaque Model

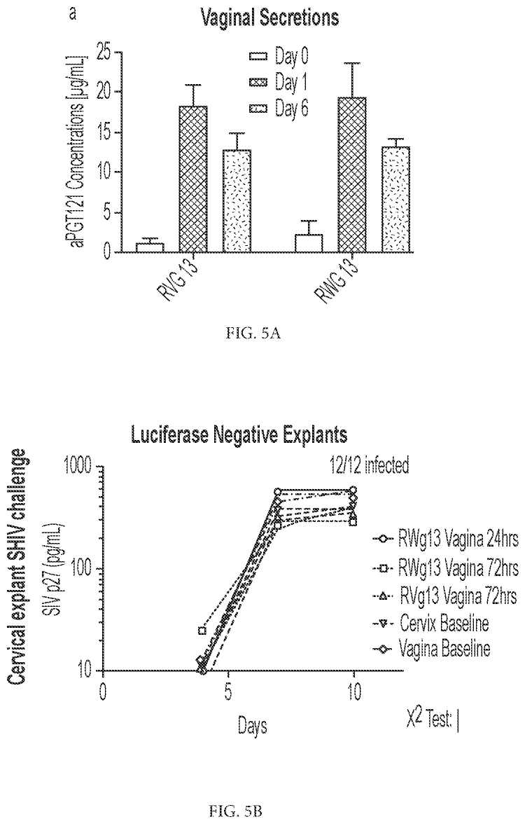

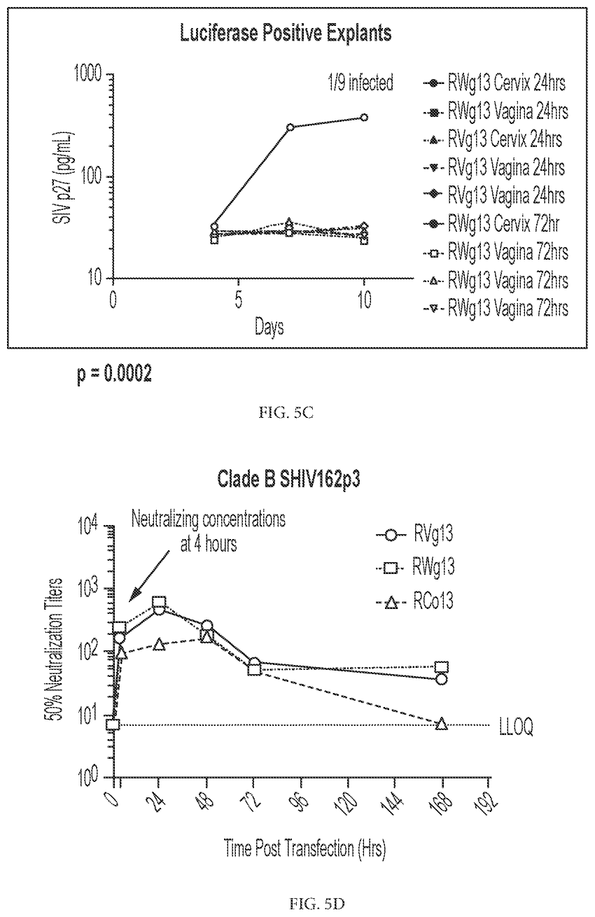

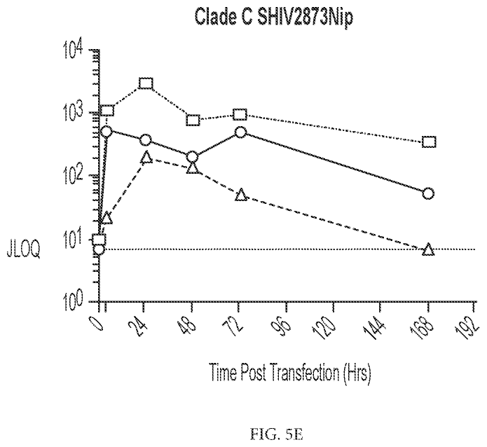

In addition, this approach was demonstrated in macaques (FIGS. 5A-5E). It was shown that at day 1 and day 6 post-delivery of mRNA encoding the anchored version of the heavy and light chain of PGT121, that −19 ug/ml of Ab was measured in the secretions at day 1 and −13 ug/ml at day 6, using a dose of 125 ug of mRNA (FIG. 5A). This dose is approximately 6× lower than the dose in sheep. Even with that low dose, 8/9 biopsies that were nanoluc+, were also resistant to an ex-vivo SHIV infection (FIGS. 5B-5E). Neutralization titers were also measured and found that neutralizing Ab titers occurred at 4 hrs post mRNA delivery (first sampling point).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A recombinant genetic construct encoding an antibody or antigen binding fragment that specifically binds to a sperm antigen and a membrane anchor, comprising a nucleic acid sequence encoding a heavy chain encoded by a nucleic acid having 95%, 99%, or 100% sequence identity to SEQ ID NO:2, a light chain encoded by a nucleic acid having 95%, 99%, or 100% sequence identity to SEQ ID NO:5, and a nucleic acid encoding a GPI membrane anchor having 95%, 99%, or 100% sequence identity to SEQ ID NO:3.

2. The recombinant genetic construct of claim 1, wherein the construct is an mRNA construct.

3. The recombinant genetic construct of claim 1, further comprising a signal sequence encoded by a nucleic acid having 95%, 99%, or 100% sequence identity to SEQ ID Nos:1 or 4.

4. The recombinant genetic construct of claim 1, wherein the antibody or an antigen binding fragment thereof comprises a heavy chain encoded by a nucleic acid sequence having 95%, 99%, or 100% sequence identity to SEQ ID NO:2, a GPI membrane anchor encoded by a nucleic acid sequence having 95%, 99%, or 100% sequence identity to SEQ ID NO:3, and a light chain encoded by a nucleic acid sequence having 95%, 99%, or 100% sequence identity to SEQ ID NO:5.

5. The recombinant genetic construct of claim 1, wherein the antibody or antigen binding fragment further comprising a heavy chain encoded by an amino acid sequence having 95%, 99%, or 100% sequence identity to SEQ ID NO:7, a GPI membrane anchor encoded by an amino acid sequence having 95%, 99%, or 100% sequence identity to SEQ ID NO:8, and a light chain encoded by an amino acid sequence having 85%, 90%, 95%, 99%, or 100% sequence identity to SEQ ID NO:10.

6. The recombinant genetic construct of claim 4, wherein the the antibody binding fragment binds to a monoclonal antibody.

7. A non-hormonal pharmaceutical contraception composition comprising:
a recombinant genetic construct encoding an antibody or antigen binding fragment thereof that specifically binds to a sperm antigen and a membrane anchor; and
an excipient.

8. The composition of claim 7, wherein the recombinant genetic construct comprises is the construct of claim 1.

9. The composition of claim 7, wherein the sperm antigen is CD52.

10. The composition of claim 7, wherein the excipient is water.

11. The composition of claim 7, wherein the sperm antigen is selected from the group consisting of sperm adhesion molecule 1 (SPAM 1), metalloprotease disintegrin cysteine (MDC), sperm protein (SP-10), fertilization antigen (FA-1), SP-17, NZ-1, NZ-2, lactate dehydrogenase (LDH-C4), sperm agglutination antigen (SAGA-1), YLP-12 peptide, human equatorial segment protein (hESP), BS-17, rabbit sperm membrane protein-B (rSMP-B), sperm acrosomal membrane-associated protein (SAMP-32), and 80 kDa human sperm antigen (HSA). In other embodiments, the antibody or antigen-binding fragment thereof binds to dorsal head and equatorial (DE), epididymal protease inhibitor (Eppin), and sperm flagella protein (SFP-2).

12. The composition of claim 7, wherein the membrane anchor contains transmembrane domains, glycosylphosphatidylinositol anchors, or myristoylation motifs.

13. The composition of claim 7, wherein the antibody or antigen binding fragment is encoded by a nucleic acid construct encoding an antibody or antigen binding fragment thereof that specifically binds to a sperm antigen and a membrane anchor.

14. The construct of claim 13, wherein the construct is mRNA.

15. (canceled)

16. A method for providing contraception to a female subject in need thereof comprising the steps of:
transfecting epithelial cells of the subject's reproductive tract with a nucleic acid construct encoding an antibody or an antigen-binding fragment that specifically binds to a sperm antigen and also encodes a membrane anchor;
administering to the subject's female reproductive tract the epithelial cells transfected with the nucleic acid construct in an amount effective to provide contraception.

17. The method of claim 16, wherein the nucleic acid construct is the construct of claim 1.

18. (canceled)

19. The method of claim 16, wherein the nucleic acid construct is delivered as an aerosol.

20. The method of claim 16, wherein the sperm antigen is CD52g.

21. The method of claim 16, wherein the subject is human.

22-27. (canceled)

28. The method of claim 19, wherein the aerosol delivery device is an atomizer or a dual-chamber syringe containing lyophilized mRNA and water and an atomizer suitable for self-insertion into the FRT.

29-30. (canceled)
